(19) United States
(12) Patent Application Publication
(10) Pub. No.: US 2026/0183142 A1
(43) Pub. Date: Jul. 2, 2026

(54) METHOD OF CONTROLLING COOLING RATE

(71) Applicant: RECENSMEDICAL, INC., Hwaseong-si (KR)

(72) Inventors: Youngsub LEE, Yongin-si (KR); Kyongkwan RO, Hwaseong-si (KR)

(21) Appl. No.: 19/548,071

(22) Filed: Feb. 24, 2026

Related U.S. Application Data
(63) Continuation of application No. PCT/KR2024/012846, filed on Aug. 28, 2024.

(30) Foreign Application Priority Data
Aug. 31, 2023 (KR) 10-2023-0115199

(51) Int. Cl. A61F 7/00

(52) U.S. Cl. CPC A61F 7/0085; A61F 7/007; A61F 2007/0052; A61F 2007/0063; A61F 2007/0086; A61F 2007/0093; A61F 2007/0096

(57) ABSTRACT

Proposed is a method of controlling the cooling rate by controlling a refrigerant spraying device configured to spray a refrigerant on a target region. The method controls a time taken for a surface temperature of the target region to reach a target cooling temperature step by step by controlling heat energy applied to the sprayed refrigerant. The method may be designed using a plurality of control modes that are varied in pattern of applying heat energy to the refrigerant.

### CROSS REFERENCE TO RELATED APPLICATIONS

This is a continuation application of International Patent Application No. PCT/KR2024/012846 filed on Aug. 28, 2024, which claims priority to Korean Patent Application No. 10-2023-0115199, filed Aug. 31, 2023, the entire contents of each of which are incorporated herein for all purposes by this reference.

### BACKGROUND

### Technical Field

The present disclosure relates generally to a method of controlling a cooling rate when cooling a target region. More specifically, the present disclosure relates to a method of controlling a rate at which a target region is cooled when spraying a refrigerant on the target region using a refrigerant spraying device.

### Description of Related Technology

Recently, there has been great interest in developing a technology that performs cooling by spraying a refrigerant on a target region. Specifically, a technology for spraying a refrigerant into a target region to cool the target region to a specific target cooling temperature creates a skin beauty effect and can be used in a variety of applications, such as producing a pain-relieving effect when used in conjunction with cosmetic procedures such as laser irradiation, Botox, and filler injection. Thus, research and development on this are being actively conducted.

### SUMMARY

One aspect is a control method of controlling a temperature of a sprayed refrigerant when spraying the refrigerant on a target region.

Another aspect is a method of dividing a temperature control method of controlling a temperature of a refrigerant sprayed on a target region into a plurality of control modes and selecting one control mode among the plurality of control modes.

Another aspect is a method of controlling an initial cooling rate based on a time point at which refrigerant spraying is started when spraying a refrigerant on a target region.

Another aspect is a method in which in first spraying for spraying a refrigerant on a first target region and in second spraying for spraying the refrigerant on a second target region, a refrigerant temperature control mode in the second spraying is determined by considering a refrigerant temperature control mode in the first spraying.

The aspects of the present disclosure are not limited to those mentioned above, and other aspects not mentioned will be clearly understood by those skilled in the art from the following description and the accompanying drawings.

Another aspect is a control method for controlling a refrigerant spraying device such that a surface temperature of a target region becomes a target temperature by cooling the target region by using the refrigerant spraying device: wherein the refrigerant spraying device comprises a temperature controller configured to generate a heat energy according to one of a plurality of control modes, wherein the control method comprises: controlling the temperature controller as a first control mode for a first time period, wherein, in the first time period, the refrigerant spraying device sprays a refrigerant to a first target region from a first time point to a second time point upon a first manipulation input is received; obtaining a surface temperature of the first target region corresponding to the first time point as a first temperature; obtaining a surface temperature of the first target region corresponding to a time point elapsed an initial time from the first time point, as a second temperature; calculating a first initial cooling rate by using the first temperature, the second temperature, and the initial time; and controlling the temperature controller for a second time period starting after an end of the first time period, wherein, in the second time period, the refrigerant spraying device sprays a refrigerant to a second target region from a third time point to a fourth time point upon a second manipulation input is received; i) wherein the temperature controller is controlled according to a second control mode in the second time period when a magnitude of the first initial cooling rate is less than a critical value, wherein a heat energy that the temperature controller controlled according to the first control mode provides until a surface temperature of the first target region reaches the target temperature in the first time period is, greater than a heat energy that the temperature controller controlled according to the second control mode provides until a surface temperature of the second target region reaches the target temperature in the second time period, ii) wherein the temperature controller is controlled according to a first control mode in the second time period when a magnitude of the first initial cooling rate is greater than a critical value.

Another aspect is a control method for controlling a refrigerant spraying device such that a surface temperature of a target region becomes a target temperature by cooling the target region by using the refrigerant spraying device: wherein the refrigerant spraying device comprises a temperature controller configured to generate a heat energy according to one of a plurality of control modes, wherein the control method comprises: controlling the temperature controller as a first control mode for a first time period, wherein, in the first time period, the refrigerant spraying device sprays a refrigerant to a first target region from a first time point to a second time point upon a first manipulation input is received; obtaining a surface temperature of the first target region corresponding to the first time point as a first temperature; obtaining a surface temperature of the first target region corresponding to a time point elapsed an initial time from the first time point, as a second temperature; calculating a first initial cooling rate by using the first temperature, the second temperature, and the initial time; and controlling the temperature controller for a second time period starting after an end of the first time period, wherein, in the second time period, the refrigerant spraying device sprays a refrigerant to a second target region from a third time point to a fourth time point upon a second manipulation input is received; i) wherein the temperature controller is controlled according to a second control mode in the second time period when a magnitude of the first initial cooling rate is less than a critical value, wherein a first standby time from the third time point to a time point when electric power is applied to the temperature controller in the second time period is, longer than a second standby time from the first time point to a time point when electric power is applied to the temperature controller in the first time period, and ii) wherein the temperature controller is controlled according to a first control mode in the second time period when a magnitude of the first initial cooling rate is greater than a critical value.

The solution for problem is not limited to the above-mentioned solution, and the solution not mentioned can be clearly understood by those skilled in a technical field including this invention from this disclosure and the attached drawings.

According to one embodiment, a surface temperature of a target region may reach a target cooling temperature relatively quickly.

According to another embodiment, the surface temperature of the target region may not reach a threshold temperature at which irreversible damage occurs.

According to another embodiment, a time taken for the temperature of the target region to reach the target cooling temperature may decrease in second spraying for spraying a refrigerant on a second target region compared to a first spraying for spraying the refrigerant on a first target region.

The effects of the present disclosure are not limited to those mentioned above, and other effects not mentioned will be clearly understood by those skilled in the art from the following description and the accompanying drawings.

### BRIEF DESCRIPTION OF THE DRAWINGS

FIG. 1 is a view illustrating a refrigerant spraying system according to an embodiment;

FIG. 2 is a view illustrating the configuration of a refrigerant spraying device according to an embodiment;

FIG. 3 is a graph illustrating how a surface temperature of a target region is varied when a refrigerant temperature is controlled according to a specific control method according to an embodiment;

FIG. 4 is a view illustrating a case where a surface temperature of a target region reaches a temperature that causes irreversible damage when cooling the target region according to an embodiment;

FIG. 5 is a view illustrating a method of designing a temperature control method according to an embodiment;

FIG. 6 is a view illustrating how a surface temperature of a target region is varied depending on the type of control mode according to an embodiment, in which (a) of FIG. 6 is a graph illustrating how a surface temperature is varied with time for each type of control mode, and (b) of FIG. 6 is a graph illustrating an initial cooling rate for each type of control mode;

FIGS. 7 and 8 are flowcharts illustrating a method of controlling a cooling rate according to an embodiment; and

FIG. 9 is a flowchart illustrating a control method of a refrigerant spraying device when a reset condition is satisfied according to an embodiment.

### DETAILED DESCRIPTION

One of the important criteria for determining the efficiency of the technology for cooling a target region is the cooling rate. In other words, it is known that as the time taken for the temperature of the target region to reach the specific target cooling temperature by cooling the target region decreases, the efficiency increases. In refrigerant spraying devices, a high cooling rate directly leads to competitiveness.

However, in refrigerant spraying through a refrigerant spraying device, when excessively large cooling energy is applied to the target region to increase the cooling rate, irreversible damage to the target region may occur. In particular, the variation in cooling energy delivered to the target region may be large depending on the temperature of a space where a procedure is performed, the temperature of the refrigerant spraying device itself, or the internal pressure of a refrigerant storage container.

In the present disclosure, there is proposed a refrigerant spraying device that cools a target region so that a temperature of the target region reaches a target cooling temperature relatively quickly while preventing excessive cooling energy from being applied to the target region, thereby providing both rapidity and safety.

According to an embodiment, there is provided a control method for controlling a refrigerant spraying device such that a surface temperature of a target region becomes a target temperature by cooling the target region by using the refrigerant spraying device: wherein the refrigerant spraying device comprises a temperature controller configured to generate a heat energy according to one of a plurality of control modes, wherein the control method comprises: controlling the temperature controller as a first control mode for a first time period, wherein, in the first time period, the refrigerant spraying device sprays a refrigerant to a first target region from a first time point to a second time point upon a first manipulation input is received; obtaining a surface temperature of the first target region corresponding to the first time point as a first temperature; obtaining a surface temperature of the first target region corresponding to a time point elapsed an initial time from the first time point, as a second temperature; calculating a first initial cooling rate by using the first temperature, the second temperature, and the initial time; and controlling the temperature controller for a second time period starting after an end of the first time period, wherein, in the second time period, the refrigerant spraying device sprays a refrigerant to a second target region from a third time point to a fourth time point upon a second manipulation input is received; i) wherein the temperature controller is controlled according to a second control mode in the second time period when a magnitude of the first initial cooling rate is less than a critical value, wherein a heat energy that the temperature controller controlled according to the first control mode provides until a surface temperature of the first target region reaches the target temperature in the first time period is, greater than a heat energy that the temperature controller controlled according to the second control mode provides until a surface temperature of the second target region reaches the target temperature in the second time period, ii) wherein the temperature controller is controlled according to a first control mode in the second time period when a magnitude of the first initial cooling rate is greater than a critical value.

A temperature of the temperature controller controlled according to the second control mode during the initial time from the third time point is, lower than a temperature of the temperature controller controlled according to the first control mode during the initial time from the first time point.

The temperature controller includes a thermoelectric element generating a heat energy upon receiving electric power, and wherein an amount of electric power per a unit time applied to the temperature controller in the first time period has a greater value than an amount of electric power per a unit time applied to the temperature controller in the second time period.

A magnitude of a second initial cooling rate calculated by using a surface temperature of the second target region corresponding to the third time point, a surface temperature of the second target region corresponding to a time point the initial time elapsed from the third time point, and the initial time has greater value than a magnitude of the first initial cooling rate.

When the temperature controller is controlled according to the second control mode in the second time period, a time consumed for a surface temperature of the first target region corresponding to the first time point to first reach the target cooling temperature in the first time period is, longer than a time consumed for a surface temperature of the second target region corresponding to the third time point to first reach the target cooling temperature in the second time period.

The initial time is determined less than or equal to 1 second.

When a magnitude of the first initial cooling rate is greater than the critical value, the temperature controller is controlled according to the first control mode in the second time period.

When a magnitude of the first initial cooling rate is less than the critical value but the second manipulation input is not received in a critical time from the second time point of the first time period, the temperature controller is controlled according to the first control mode in the second time period.

The critical time is determined less than or equal to 60 seconds.

The control method comprising spraying, according to a third manipulation input, a refrigerant to a third target region in a third time period which is from a fifth time point after the fourth time point to a sixth time point; wherein when a magnitude of the first initial cooling rate is less than the critical value and a magnitude of the second initial cooling rate is less than the critical value, the temperature controller is controlled according to a third control mode in the third time period, and wherein a time consumed for a surface temperature of the third target region corresponding to the fifth time point to first reach the target cooling temperature in the third time period is, shorter than a time consumed for a surface temperature of the second target region corresponding to the third time point to first reach the target cooling temperature in the second time period.

A heat energy that the temperature controller controlled according to the second control mode provides from the second time point to a time point that a surface temperature of the second target region reaches the target cooling temperature in the second time period is, greater than a heat energy that the temperature controller controlled according to the third control mode provides from the fifth time point to a time point a surface temperature of the second target region reaches the target temperature in the third time period.

The temperature controller includes a thermoelectric element generating a heat energy upon receiving electric power, and wherein when the temperature controller is controlled according to the second control mode in the second time period and is controlled according to the third control mode in the third time period, an amount of electric power per a unit time applied to the temperature controller in the second time period has a greater value than an amount of electric power per a unit time applied to the temperature controller in the third time period.

When a magnitude of the first initial cooling rate is less than the critical value and a magnitude of the second initial cooling rate is greater than the critical value, the temperature controller is controlled according to the second control mode in the third time period.

When a magnitude of the first initial cooling rate is less than the critical value, a magnitude of the second initial cooling rate is less than the critical value, but the third manipulation input is not received in a critical time from the fourth time point of the second time period, the temperature controller is controlled according to the first control mode in the third time period.

A refrigerant spray started at the first time point according to received the first manipulation input is a refrigerant spray performed firstly after a power of the refrigerant spraying device is on.

According to other embodiment, there is provided a control method for controlling a refrigerant spraying device such that a surface temperature of a target region becomes a target temperature by cooling the target region by using the refrigerant spraying device: wherein the refrigerant spraying device comprises a temperature controller configured to generate a heat energy according to one of a plurality of control modes, wherein the control method comprises: controlling the temperature controller as a first control mode for a first time period, wherein, in the first time period, the refrigerant spraying device sprays a refrigerant to a first target region from a first time point to a second time point upon a first manipulation input is received; obtaining a surface temperature of the first target region corresponding to the first time point as a first temperature; obtaining a surface temperature of the first target region corresponding to a time point elapsed an initial time from the first time point, as a second temperature; calculating a first initial cooling rate by using the first temperature, the second temperature, and the initial time; and controlling the temperature controller for a second time period starting after an end of the first time period, wherein, in the second time period, the refrigerant spraying device sprays a refrigerant to a second target region from a third time point to a fourth time point upon a second manipulation input is received; i) wherein the temperature controller is controlled according to a second control mode in the second time period when a magnitude of the first initial cooling rate is less than a critical value, wherein a first standby time from the third time point to a time point when electric power is applied to the temperature controller in the second time period is, longer than a second standby time from the first time point to a time point when electric power is applied to the temperature controller in the first time period, and ii) wherein the temperature controller is controlled according to a first control mode in the second time period when a magnitude of the first initial cooling rate is greater than a critical value.

The above-described objectives, features, and advantages of the present disclosure will be more clearly understood from the following detailed description when taken in conjunction with the accompanying drawings. The present disclosure may, however, be embodied in many different forms and should not be limited to the exemplary embodiments. Hereinafter, reference will now be made in more detail to embodiments which are illustrated in the accompanying drawings.

In the drawings, the thicknesses of layers and regions are exaggerated for clarity. Further, when an element or layer is referred to as being “on” or “above” another element or layer, it can be directly above the other element or layer or intervening elements or layers may be present therebetween. In principle, like reference numerals designate like elements throughout the specification. In addition, like reference numerals are used to designate elements which have the same function within the same idea illustrated in the drawings of each embodiment, and overlapping descriptions thereof will be omitted.

Numerals (e.g., first, second, etc.) used in the description herein are merely identifiers for distinguishing one element from another element.

In addition, the element terms “module” and “part” used in the following embodiments are given or mixed together only considering the ease of creating the specification, and have no meanings or roles that are distinguished from each other by themselves.

As used herein, the singular forms re intended to include the plural forms as well, unless the context clearly indicates otherwise.

In the following embodiments, the terms “comprise”, “include”, “have”, etc. when used in this specification, specify the presence of stated features or elements, but do not preclude the presence or addition of one or more other features or elements.

In the drawings, the sizes of elements may be exaggerated or reduced for convenience of explanation. For example, the sizes and thicknesses of elements in the drawings are arbitrarily illustrated for convenience of explanation, and embodiments of the present disclosure are not limited thereto.

When some example embodiments may be embodied otherwise, specific process steps described herein may be performed otherwise. That is, for example, two process steps described in a sequential order may be performed around the same time, or in reverse order.

In the following embodiments, when films, regions, elements, etc. are referred to as being connected to each other, they can be directly connected or be indirectly connected to each other with one or more intervening films, regions, elements, etc. interposed therebetween.

For example, when films, regions, elements, etc. are referred to as being electrically connected to each other, they can be electrically directly connected or be electrically indirectly connected to each other with one or more intervening films, regions, elements, etc. interposed therebetween.

In the following embodiments, when films, regions, elements, etc. are referred to as being fluidly connected to each other, it may be understood that they may form at least a part of a flow path through which each fluid flows.

For example, that an element A is fluidly connected to an element B may mean that a fluid passing through a flow path formed by the element A may reach a flow path formed by the element B or vice versa. Specifically, when the element A and the element B are coupled and the flow path formed by the element A and the flow path formed by the element B are directly connected to each other, the element A and the element B can be considered to be fluidly connected to each other. Otherwise, when the element A and element B are connected to each other through an element C, such as a conduit, so that the flow path formed by the element A and the flow path formed by the element B are indirectly connected to each other through a flow path formed by the element C, the element A and the element B can be considered to be fluidly connected to each other. Here, the element C can be understood as fluidly connecting the elements A and B. Furthermore, the element A and the element B may be fluidly connected to each other through a plurality of elements.

The present disclosure relates to a method of controlling a cooling rate in cooling a target region. More specifically, the present disclosure relates to a method of cooling a target region by spraying a refrigerant onto the target region using a refrigerant spraying device, and controlling a rate at which a surface temperature of the target region reaches a target cooling temperature. Furthermore, the present disclosure is to control a temperature of a refrigerant sprayed on a target region so that a surface temperature of the target region does not decrease excessively during cooling of the target region.

The “target region” may refer to a region of a target where a refrigerant is sprayed. The “target” may refer to a body part for which a cosmetic or medical effect is desired to occur through a procedure, treatment, or care. Hereinafter, the target will be referred to as the skin, and the target region will accordingly be described as a region on the skin surface where the refrigerant is sprayed, but the technical idea of the present disclosure is not limited thereto.

The “surface temperature” of the target region may refer to a temperature measured for the target region using a temperature sensor. Specifically, the surface temperature of the target region may refer to a temperature measured for a temperature sensing region that at least partially overlaps the target region.

The “refrigerant” may refer to a substance that can apply cooling energy to the target region. The refrigerant may include, for example, carbon dioxide (CO₂), liquid nitrogen (LN), nitrogen dioxide (NO₂), nitrogen monoxide (NO), hydrofluorocarbon (HFC)-based materials, methane (CH₄), PFC, SF₆, cooling water, cooling gas, or a combination thereof.

### Refrigerant Spraying System

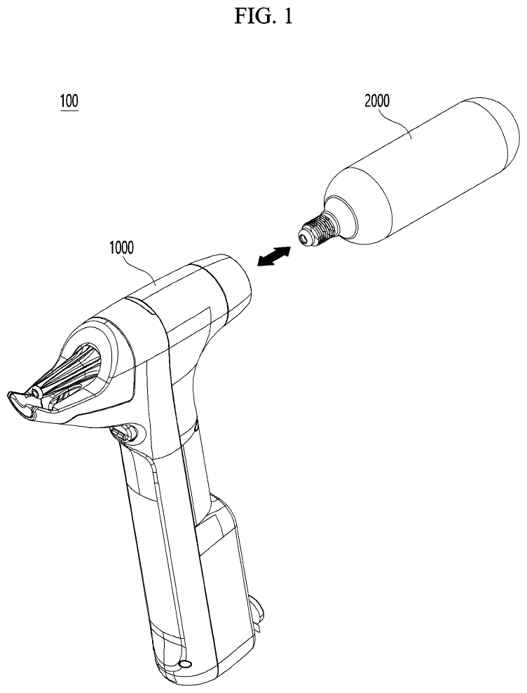

Hereinafter, a refrigerant spraying system 100 will be described with reference to FIG. 1.

FIG. 1 is a view illustrating a refrigerant spraying system 100 according to an embodiment. Referring to FIG. 1, the refrigerant spraying system 100 may include a refrigerant spraying device 1000 and a cartridge 2000.

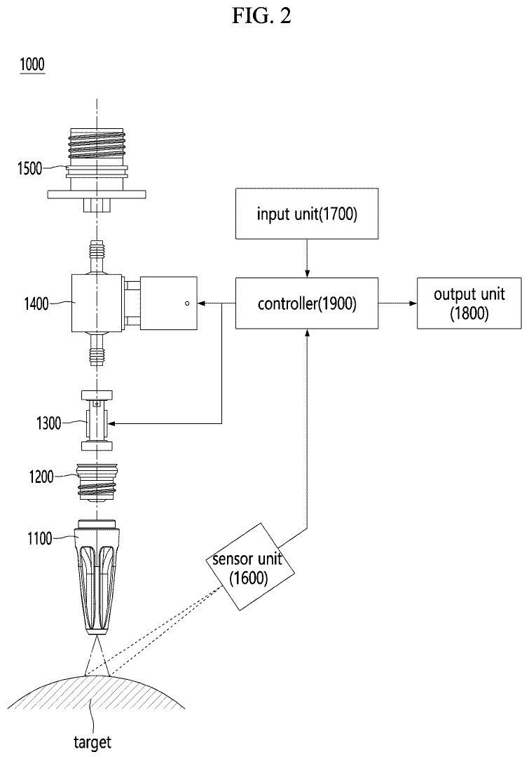

The refrigerant spraying device 1000 may be designed to receive a refrigerant and spray the refrigerant. Specifically, as illustrated in FIG. 1, the cartridge 2000 storing the refrigerant may be coupled to the refrigerant spraying device 1000, and the refrigerant stored in the cartridge 2000 may be sprayed to the outside through the refrigerant spraying device 1000. The configurations of the refrigerant spraying device 1000 for spraying the refrigerant will be described in detail later with reference to FIG. 2.

The refrigerant spraying device 1000 may control a flow rate and temperature of the supplied refrigerant. Specifically, the refrigerant spraying device 1000 may control a supply amount, supply time, temperature, and/or pressure of the refrigerant.

The cartridge 2000 may store the refrigerant therein. The cartridge 2000 may store the refrigerant under a predetermined pressure, and the pressure may be determined between about 35 bar and about 100 bar at a temperature of 0° C. to 40° C.

The pressure inside the cartridge 2000 may vary depending on a temperature of the cartridge 2000. For example, when the cartridge 2000 is stored at a relatively high temperature, the temperature of the cartridge 2000 may increase, and the pressure inside the cartridge 2000 may also increase accordingly.

The pressure inside the cartridge 2000 affects a pressure of the refrigerant before it is sprayed from the refrigerant spraying device 1000, and thereby affects an amount of temperature change caused by expansion when the refrigerant is sprayed from the refrigerant spraying device 1000. Specifically, as the pressure inside the cartridge 2000 increases, the pressure of the refrigerant inside the refrigerant spraying device 1000 may increase, thereby increasing a spray velocity of the refrigerant, and the temperature of the refrigerant sprayed from the refrigerant spraying device 1000 may decrease according to the law of energy conservation.

Meanwhile, in FIG. 1, the cartridge 2000 is illustrated as being coupled to the refrigerant spraying device 1000, but the technical idea of the present disclosure is not limited thereto. For example, the refrigerant spraying device 1000 may receive the refrigerant from a storage where the refrigerant is stored, such as a tank, through a hose or the like.

### Configuration of Refrigerant Spraying Device

Hereinafter, the configurations of the refrigerant spraying device 1000 will be described with reference to FIG. 2.

FIG. 2 is a view illustrating the configuration of a refrigerant spraying device 1000 according to an embodiment.

Referring to FIG. 2, the refrigerant spraying device 1000 may include a refrigerant spraying unit 1100, a spraying unit coupler 1200, a temperature controller 1300, a flow regulator 1400, a cartridge coupler 1500, a sensor unit 1600, an input unit 1700, an output unit 1800, and a controller 1900.

The refrigerant spraying unit 1100 may include a structure for spraying the refrigerant. Specifically, the refrigerant spraying unit 1100 may extend from a first end to a second end thereof to form a flow path, and the flow path may have a portion that is relatively narrow in width. A fluid passing through the refrigerant spraying unit 1100 expands as its pressure decreases while passing through the narrow portion, resulting that the fluid is sprayed at high velocity. At this time, the fluid expands adiabatically while passing through the refrigerant spraying unit 1100 and its temperature decreases. The refrigerant may be controlled to a temperature suitable for a procedure or treatment by the temperature controller 1300, which will be described later.

The refrigerant spraying unit 1100 may be understood as a nozzle. However, the technical idea of the present disclosure is not limited thereto, and the refrigerant spraying unit 1100 may be understood as a configuration including a tubular fluid movement path with a narrow width.

The refrigerant spraying unit 1100 may be detachably attached to the refrigerant spraying device 1000. For example, the refrigerant spraying unit 1100 may be coupled to the refrigerant spraying device 1000 through the spraying unit coupler 1200 or may be removed from the refrigerant spraying device 1000. Alternatively, the refrigerant spraying unit 1100 may be physically connected to and integrated with the refrigerant spraying device 1000.

The refrigerant spraying unit 1100 may be coupled to the spraying unit coupler 1200. The spraying unit coupler 1200 may have a flow path formed therein for movement of the refrigerant. For example, the spraying unit coupler 1200 may include a discharge hole, and the refrigerant may be moved to the refrigerant spraying unit 1100 coupled to the spraying unit coupler 1200 through the discharge hole.

The temperature controller 1300 may control a temperature of the refrigerant. For example, the temperature controller 1300 may increase the temperature of the refrigerant by providing heat energy to the refrigerant, and the temperature of the refrigerant may be controlled depending on an amount of heat energy provided by the temperature controller 1300. The refrigerant sprayed through the refrigerant spraying unit 1100 may have a relatively low temperature as described above, and in this case, the temperature of the refrigerant may vary depending on the heat energy provided by the temperature controller 1300.

The temperature controller 1300 may include a heat generating unit that generates heat energy and a heat transfer unit that transfers the generated heat energy to a flow path for movement of the refrigerant.

As an example, the heat generating unit may be a thermoelectric element. Specifically, the heat generating unit may include a first surface and a second surface, and according to a thermoelectric effect such as Peltier's effect, the first surface may generate heat while the second surface may absorb heat when power is applied. Here, the heat energy generated as the first surface generates heat may be proportional to voltage, current, square of voltage, or power applied to the heat generating unit. In other words, as a voltage or current applied to the heat generating unit increases, an amount of heat energy generated may also increase.

As an example, the heat transfer unit may be provided in at least a portion of a tube through which the refrigerant is moved in the refrigerant spraying device 1000. Specifically, the heat transfer unit may include a porous structure or microtubular structure installed inside the tube, and direct heat exchange may occur between the heat transfer unit and the refrigerant as the refrigerant passes through the porous structure.

The flow regulator 1400 may control movement of the refrigerant. For example, the flow controller 1400 may include a valve, and may open and close the valve in response to a signal received from the controller 1900. Depending on whether the valve is opened or closed, the refrigerant may or may not be moved. A flow rate of the refrigerant may be controlled depending on an opening ratio of the valve.

An amount of cooling energy per unit time provided to the target region may be controlled using the flow regulator 1400. For example, when the flow controller 1400 is a valve, an amount of refrigerant sprayed per unit time to the target region may be controlled when an opening and closing cycle of the valve is controlled. As the amount of refrigerant per unit time sprayed into the target region is controlled, the cooling energy per unit time delivered to the target region may also be controlled, and a rate of change of a surface temperature of the target region may be controlled accordingly.

The cartridge coupler 1500 may accommodate at least a portion of the above-described cartridge 2000. With the cartridge 2000 coupled to the cartridge coupler 1500, the refrigerant stored in the cartridge 2000 may be moved to the refrigerant spraying device 1000.

The sensor unit 1600 may measure a temperature of a region where the refrigerant is sprayed. For example, the sensor unit 1600 may measure a temperature of a target region where the refrigerant is sprayed and provide measurement information to the controller 1900.

The input unit 1700 may receive a user's input. For example, the input unit 1700 may include at least one push button switch and provide a push input signal to the controller 1900 in response to the user's pressure on the switch, and the controller 1900 may control opening and closing of the flow regulator 1400 based on the push input signal.

In addition, the input unit 1700 may include at least one rotary switch and provide a rotation input signal to the controller 1900 in response to the manipulation, and the controller 1900 may set a target cooling temperature or target cooling time based on the rotation input signal. Here, the target cooling temperature refers to a temperature at which a target to which the refrigerant is to be sprayed, for example, a target region, is to be cooled. In addition, the target cooling time refers to a time during which spraying of the refrigerant needs to be maintained or a time should be elapsed after the surface temperature of the target region reaches the target cooling temperature.

The output unit 1800 may output an interface for use of the refrigerant spraying device 1000 and various information to the user. For example, the output unit 1800 may include a display, and the output unit 1800 may output an interface for setting the target cooling temperature or target cooling time through the display, and while the refrigerant spraying device 1000 is operated, may output information such as a real-time temperature of the target region measured by the sensor unit 1600 or a total time during which the refrigerant is sprayed.

The controller 1900 may control the configurations of the refrigerant spraying device 1000. For example, the controller 1900 may control the temperature of the sprayed refrigerant by controlling the temperature controller 1300, control the flow of the refrigerant by controlling the flow regulator 1400, and output specific information to the user by controlling the output unit 1800.

Although not illustrated in FIG. 2, the refrigerant spraying device 1000 may further include a memory. Various data, a program, or an application necessary for the operation of the refrigerant spraying device 1000 may be stored in the memory. The program or application stored in the memory may include one or more instructions. The various data may include information acquired from the sensor unit 1600, a temperature of the temperature controller 1300, and voltage, current, or power values applied to the temperature controller 1300. The program stored in the memory may include instructions corresponding to a plurality of control modes for controlling the temperature controller 1300, which will be described later. The various data may be temporarily or semi-permanently stored in the memory. Examples of memory may include a hard disk drive (HDD), solid state drive (SSD), flash memory, read-only memory (ROM), random access memory (RAM), and the like.

In addition, the refrigerant spraying device 1000 may include a communication unit for communicating with an external device. The communication unit may perform wired or wireless communication, and may include, for example, a wired/wireless local area Network (LAN) module, a wide area network (WAN) module, an Ethernet module, a Bluetooth module, a Zigbee module, a universal serial bus (USB) module, an IEEE 1394 module, a Wi-Fi module, a mobile communication module, a satellite communication module, or a combination thereof, but is not limited thereto.

The refrigerant spraying device 1000 may be operated in the following manner.

First, the controller 1900 may set the target cooling temperature and/or the target cooling time. For example, the controller 1900 may provide an interface that guides the user to set the target cooling temperature and the target cooling time through the output unit 1800, receive a setting input signal in response to the manipulation through the input unit 1700, and set the target cooling temperature and the target cooling time based on the received setting input signal.

Thereafter, the controller 1900 may output a message indicating to the user that operation preparation is complete through the output unit 1800, receive a switch-on input signal in response to the manipulation through the input unit 1700, and allow the refrigerant to be sprayed based on the received switch-on input signal.

While the refrigerant is sprayed, the controller 1900 may acquire a temperature value obtained by measuring the temperature of the target region where the refrigerant is sprayed through the sensor unit 1600, and control the temperature controller 1300 by comparing the acquired temperature value with the set target cooling temperature. At this time, when the acquired temperature value is lower than the target cooling temperature, the controller 1900 may increase the heat energy applied to the refrigerant through the temperature controller 1300, and when the acquired temperature value is higher than the target cooling temperature, the controller 1900 may reduce the heat energy applied to the refrigerant through the temperature controller 1300.

The operation of the refrigerant spraying device 1000 is not limited to the above-described embodiment. For example, the refrigerant spraying device 1000 may be operated by continuously providing a predetermined amount of heat energy to the refrigerant without monitoring the temperature of the target region, and thus may control the temperature of the refrigerant within a predetermined range. In this case, the step of setting or receiving input of the target cooling temperature may be omitted.

### Control Method of Refrigerant Spraying Device

Hereinafter, a control method of the refrigerant spraying device 1000 will be described. More specifically, description will be given of a method of controlling the temperature of the refrigerant sprayed on the target region so that the surface temperature of the target region becomes the target cooling temperature. The temperature of the sprayed on the target region may be controlled depending on the amount of heat energy applied to the refrigerant through the temperature controller 1300.

Hereinafter, for convenience of explanation, the temperature controller 1300 will be described as including a thermoelectric element and generating heat energy corresponding to an applied voltage value or current value, but the technical idea of the present disclosure is not limited thereto.

When the temperature controller 1300 includes the thermoelectric element, as will be described later, the heat energy applied to the refrigerant may be controlled by controlling a magnitude of voltage or current applied to the temperature controller 1300, and the temperature of the refrigerant sprayed on the target region may be controlled accordingly. As a result, the surface temperature of the target region may be controlled to a specific temperature.

### Conventional Control Method of Refrigerant Spraying Device

In controlling the temperature controller 1300 to maintain the surface temperature of the target region at the target cooling temperature, a feedback control method may be used. For example, in calculating a voltage value or current value applied to the temperature controller 1300, the following proportional integral differential (PID) control equation may be used.

$$P ( t ) = {C}_{p} error ( t ) + {C}_{i} {\int }_{0}^{t} error ( t ) dt + {C}_{d} \frac{d ( error ( t ) )}{dt}$$

Here, P(t) is a magnitude of a signal applied to the temperature controller 1300 and may refer to a voltage value, a current value, a squared voltage value, or a power value. In addition, error(t) may refer to a difference between the target cooling temperature and the surface temperature of the target region measured by the sensor unit 1600, and Cp, Ci, and Cd may each refer to a gain value or gain.

Using the above PID control equation, the voltage value or current value that needs to be applied to the temperature controller 1300 may be calculated in real time, and over time, the difference between the target cooling temperature and the surface temperature of the target region may be reduced to a negligible level.

Meanwhile, each term may be omitted from the above control equation and P control, PI control, and PD control methods may be used.

The above feedback control method may be used in the process of refrigerant spraying. For example, the voltage value or current value applied to the temperature controller 1300 may be determined through the feedback control method from a time point at which refrigerant spraying is started by the flow regulator 1400.

Here, a time taken for the surface temperature of the target region to reach the target cooling temperature, that is, a cooling rate, may be changed depending on when the feedback control method is performed. For example, when comparing a case where the temperature controller 1300 is controlled through feedback control at the time point at which refrigerant spraying is started and a case where feedback control is performed after a predetermined time elapses from the time point at which refrigerant spraying is started, the latter case may exhibit a faster cooling rate.

When the refrigerant is sprayed, as described above, the temperature of the refrigerant relatively decreases (e.g., about −78° C. to −50° C.) due to expansion of the refrigerant, so the target region may be rapidly cooled. In a case where feedback control is used at the time point at which refrigerant spraying is started, power is applied to the temperature controller 1300 based on the difference between the surface temperature of the target region and the target cooling temperature, and heat energy is transferred to the refrigerant accordingly. Therefore, the temperature of the refrigerant sprayed on the target region becomes higher than when feedback control is not performed. As a result, as a time point when the feedback control is performed becomes later from a time point at which refrigerant spraying is started, the faster the surface temperature of the target region reaches to the target cooling temperature.

As described above, in order to increase the cooling rate further, there may be proposed the following control method for controlling the temperature controller 1300. For example, no voltage or current may be applied at the beginning of refrigerant spraying, and when a feedback control start condition is satisfied, voltage or current may be applied to the temperature controller 1300 through the feedback control method.

Here, the feedback control start condition may be satisfied, for example, when a control threshold time elapses from the time point at which refrigerant spraying is started or when the surface temperature of the target region becomes equal to or less than a control threshold temperature.

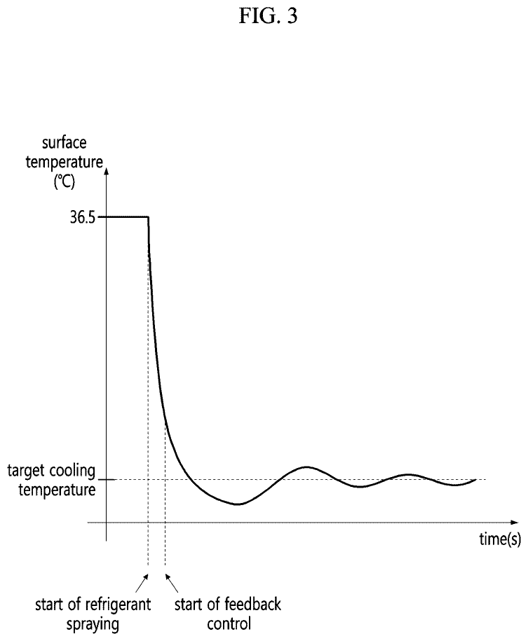

FIG. 3 illustrates how the surface temperature of the target region is varied when feedback control is performed after a predetermined time elapses from the start of refrigerant spraying. Referring to FIG. 3, from the time point at which refrigerant spraying is started to the time point at which feedback control is started, the surface temperature rapidly decreases as the refrigerant is sprayed, forming a steep curve. From the time point at which feedback control is started, the surface temperature converges to the target cooling temperature, forming a relatively gentle curve.

### Necessity of Ensuring Safety of Control

Meanwhile, the above-described control method that does not provide heat energy to the refrigerant at the beginning of refrigerant spraying (e.g., for a preset time from the time point at which refrigerant spraying is started) may cause damage to the target region in special environments.

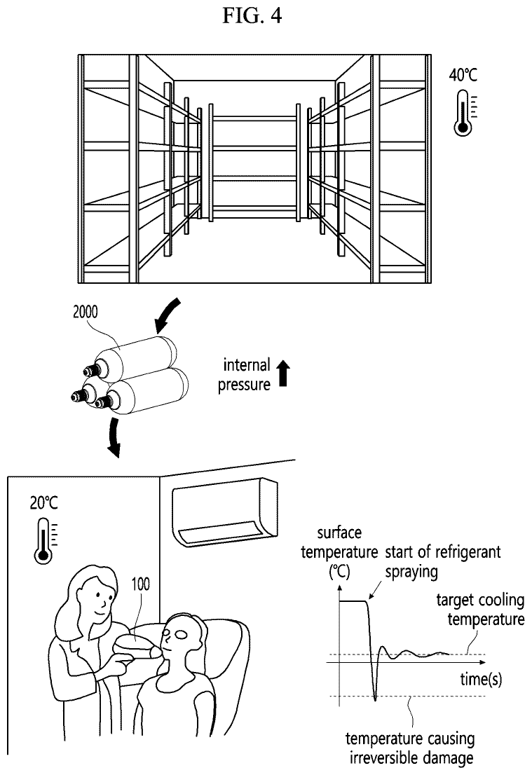

Hereinafter, problems associated with the above-described control method in special environments will be described with reference to FIG. 4.

FIG. 4 is a view illustrating a case where a surface temperature of a target region reaches a temperature that causes irreversible damage when cooling the target region according to an embodiment.

Referring to FIG. 4, a temperature of a space where the cartridge 2000 is stored may be relatively high. In addition, referring to FIG. 4, a temperature of a space where a cooling procedure is performed may be relatively low.

First, when the temperature of the space where the cartridge 2000 is stored is relatively high, an internal pressure of the cartridge 2000 may also be relatively high. As the internal pressure of the cartridge 2000 increases, the temperature of the refrigerant sprayed from the refrigerant spraying device 1000 may decrease.

When the cartridge 2000 with a high internal pressure is used and heat energy is not applied to the refrigerant for a predetermined time as described above, the refrigerant may be sprayed on the target region at an excessively low temperature. Therefore, the surface temperature of the target region may become excessively low before feedback control is started. In other words, as illustrated in FIG. 4, as the surface temperature of the target region becomes lower than a temperature that causes irreversible damage, this irreversible damage may occur in the target region.

Meanwhile, when the temperature of the space where the cooling procedure is performed is relatively low, the target region may also be excessively cooled. The refrigerant sprayed on the target region may decrease in its temperature as it expands, and then may increase in its temperature due to external air until it reaches the target region. This is because a temperature of the external air is higher than the temperature of the sprayed refrigerant, so the sprayed refrigerant comes in contact with the external air and the heat from the outside air moves to the refrigerant.

As the temperature of the space where the cooling procedure is performed decreases, that is, a difference between the temperature of the external air and the sprayed refrigerant decreases, an amount of heat transferred to the sprayed refrigerant from the external air decreases, so a relatively low temperature refrigerant may be sprayed on the target region. As described above, when the temperature of the space where the cooling procedure is performed is low, a relatively low temperature refrigerant may be sprayed on the target region compared to when the temperature thereof is high, thereby excessively decreasing the temperature of the target region may decrease excessively and causing irreversible damage.

A special environment in which the two example situations described above occur simultaneously may be considered. For example, as illustrated in FIG. 4, for reasons such as countries with hot weather or seasons with high temperatures, there may be created a special environment in which the cartridge 2000 is stored in a warehouse at about 40° C. and the space where the cooling procedure is performed is air conditioned and maintained at about 20° C. In this case, the sprayed refrigerant has a relatively low temperature due to the high internal pressure of the cartridge 2000 and the temperature difference between the sprayed refrigerant and the external air is not large. Therefore, the sprayed refrigerant may reach the target region while its temperature is maintained relatively low without increasing. As a result, as illustrated in FIG. 4, the surface temperature of the target region reaches a temperature that causes irreversible damage before the feedback control is performed, causing irreversible damage to the target region (e.g., pigmentation due to destruction of melanocytes, nerve damage, and the like).

In the above, the special environment related to the internal pressure or external temperature of the cartridge 2000 has been described. However, the technical idea of the present disclosure is not limited thereto, additional special environments in which irreversible damage may occur may be created by other variables, such as a temperature of a tube through which the refrigerant is moved in the refrigerant spraying device 1000.

Hereinafter, a control method for controlling a refrigerant temperature to prevent irreversible damage in special environments will be described.

### Step-by-Step Control Method

In order to prevent irreversible damage caused by the special environment described with reference to FIG. 4, an initial heating method of applying a predetermined level of heat energy to the refrigerant needs to be performed before feedback control is performed after the start of refrigerant spraying. By performing the initial heating method, the temperature of the refrigerant reaching the target region may be controlled to equal to or higher than a predetermined level. Therefore, an amount of change in the surface temperature of the target region for a predetermined time from the time point at which refrigerant spraying is started becomes equal to or less than a predetermined, thereby preventing the surface temperature of the target region from excessively decreasing before feedback control.

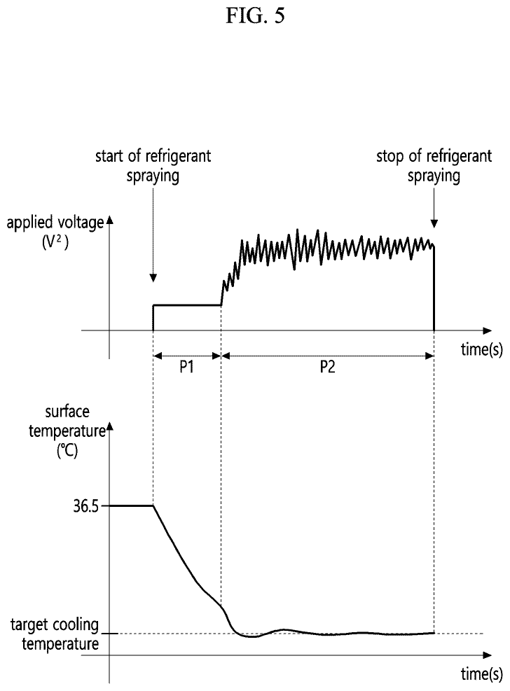

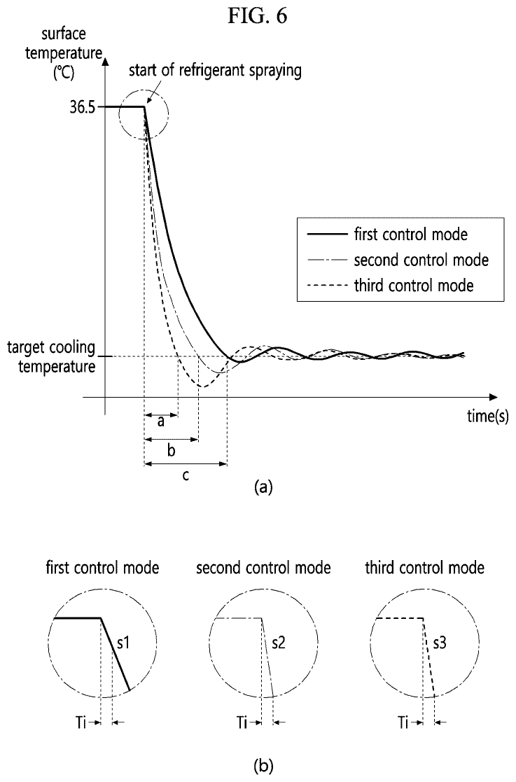

Hereinafter, a temperature control method combining the initial heating method and the feedback control method will be described in more detail with reference to FIGS. 5 and 6.

FIG. 5 is a view illustrating a method of designing a temperature control method according to an embodiment.

The temperature control method may be divided into initial heating, which applies a predetermined level of heat energy to the refrigerant from the time point at which refrigerant spraying is started until a predetermined condition is satisfied, and feedback control, which applies heat energy to the refrigerant using the above-described feedback control method. Therefore, referring to FIG. 5, a time period during which the refrigerant is sprayed may be divided into an initial heating period P1 during which initial heating is performed and a feedback control period P2 during which feedback control is performed.

In the initial heating period P1, a predetermined level of heat energy may be applied to the refrigerant by the temperature controller 1300. Specifically, the controller 1900 may apply heat energy to the refrigerant by applying voltage, current, or power to the temperature controller 1300 in a specific pattern. For example, as illustrated in FIG. 5, the controller 1900 may apply a predetermined amount of voltage or a predetermined amount of current to the temperature controller 1300 in the initial heating period P1. For another example, the controller 1900 may apply voltage or current to the temperature controller 1300 in the initial heating period P1, and a magnitude of the applied voltage or current may gradually increase according to an increasing function or may gradually decrease according to a decreasing function. For another example, the controller 1900 may not apply voltage or current to the temperature controller 1300 for a preset standby time from the start of the initial heating period P1, and may apply voltage or current after the preset standby time elapses. For another example, the controller 1900 may apply a predetermined amount of voltage or a predetermined amount of current to the temperature controller 1300 at a predetermined cycle in the initial heating period P1. There are various methods of applying voltage, current, or power to the temperature controller 1300 to apply heat energy to the refrigerant in the initial heating period P1, and are not limited to the examples described above.

The initial heating method may be performed from the time point at which refrigerant spraying is started until a predetermined condition is satisfied, and the predetermined condition may be set in the same manner as the feedback control start condition described above. For example, the initial heating method may be performed until a preset time elapses from the time point at which refrigerant spraying is started. As another example, the initial heating method may be performed until the surface temperature of the target region reaches a preset temperature.

In the feedback control period P2 after the initial heating period P1, voltage or current may be applied to the temperature controller 1300 according to feedback control. The feedback control method has already been described above and thus will be omitted.

According to the above-described initial heating method, a rate at which the surface temperature of the target region decreases as the refrigerant is sprayed may be slower compared to when the initial heating method is not performed, thereby preventing excessive cooling of the target region.

However, as the heat energy applied to the refrigerant in the initial heating period P1 increases, the time taken for the surface temperature of the target region to reach the target cooling temperature may increase. Specifically, in the initial heating period P1, heat may move from the temperature controller 1300 to the refrigerant, and the cooling rate may decrease with an increase in amount of moving heat. As a result, the cooling rate may decrease as the temperature of the refrigerant sprayed in the initial heating period P1 increases in order to prevent excessive cooling of the target region, creating a mutually exclusive relationship between safety and rapidity of procedure.

Meanwhile, the rapidity of the cooling procedure is directly related to shortening procedure time and increasing operator's convenience, and needs to be treated as equally important as the safety of the cooling procedure. Therefore, it is required to design a temperature control method that takes into account both the rapidity and safety of the cooling procedure, and a step-by-step control method will be described below with reference to FIG. 6.

Hereinafter, for convenience of explanation, it will be described that the temperature control method is a combination of the initial heating method and the feedback control method described above and the initial heating method applies a predetermined amount of voltage or a predetermined amount of current to the temperature controller 1300 for a predetermined time. However, the technical idea of the present disclosure is not limited thereto, and the temperature control method may also be applied when using other initial heating methods.

The cooling rate at which the target region is cooled may be determined depending on an amount of heat energy applied to the refrigerant in the initial heating period P1. Specifically, assuming that the cooling procedure is performed on a substantially same target in a substantially same environment, the time taken for the surface temperature of the target region to reach the target cooling temperature may increase with an increase in amount of heat energy applied to the refrigerant in the initial heating period P1, thereby decreasing the cooling rate.

Here, the amount of heat energy applied to the refrigerant may correspond to a heat generation amount of the temperature controller 1300 in the initial heating period P1. In addition, the heat generation amount of the temperature controller 1300 may correspond to an amount of power applied to the temperature controller 1300, so the amount of heat energy applied to the refrigerant in the initial heating period P1 may correspond to the amount of power applied to the temperature controller 1300.

The amount of heat energy described above may be controlled depending on an amount of voltage, current, or power applied to the temperature controller 1300 in the initial heating period P1. Specifically, as the voltage or current applied to the temperature controller 1300 in the initial heating period P1 increases and the length of the initial heating period P1 increases, the amount of heat energy applied to the refrigerant may increase.

By controlling the heat generation amount of the temperature controller 1300 in the initial heating period P1, a step-by-step control method may be designed as will be described later.

FIG. 6 is a view illustrating how a surface temperature of a target region is varied depending on the type of control mode according to an embodiment. (a) of FIG. 6 is a graph illustrating how a surface temperature is varied with time for each type of control mode, and (b) of FIG. 6 is a graph illustrating an initial cooling rate for each type of control mode.

The graph illustrated in (a) of FIG. 6 may be understood as being obtained by performing the cooling procedure on a substantially same target in a substantially same environment but through different control modes. Here, the substantially same environment may refer to an environment in which the temperature of the space where the cooling procedure is performed, the temperature of the space where the cartridge 2000 is stored before the cooling procedure, the temperature or internal pressure of the cartridge 2000 immediately before the cooling procedure, and/or the temperature of the target region before the cooling procedure are within a specific range respectively. Also, here, the substantially same target may refer to a target region within the same body part or an experimental artificial skin manufactured substantially the same.

Referring to FIG. 6, a first control mode, a second control mode, and a third control mode may be designed depending on how much heat energy is applied in the initial heating period P1.

The first to third control modes may be understood as control modes in which the initial heating method and the feedback control method are performed, but at least one of the magnitude of voltage, current, or power and the feedback control start condition is different.

First, the first control mode may exhibit a larger voltage, current, or power applied to the temperature controller 1300 in the initial heating period P1 than the second control mode or the third control mode. Alternatively, the first control mode may exhibit a larger amount of heat energy applied to the refrigerant per unit time than the second control mode or the third control mode. Therefore, when performing the cooling procedure on the substantially same target in the substantially same environment, an initial cooling rate in the first control mode may lower than that in the second control mode or the third control mode.

Here, the initial cooling rate may refer to an average cooling rate for a short period of time from the time point at which refrigerant spraying is started. Specifically, the initial cooling rate may refer to a value obtained by dividing a difference between a first surface temperature of the target region at a first time point at which refrigerant spraying is started and a second surface temperature of the target region at a second time point after a preset initial time Ti elapses from the first time point by the initial time Ti.

Here, the initial time Ti refers to a time for calculating the initial cooling rate. Therefore, the initial time Ti may be relatively short. For example, the initial time Ti may be shorter than a time taken for the surface temperature of the target region to reach the target cooling temperature (e.g., −5° C. to 5° C.) after refrigerant spraying is started in a controlled environment (e.g., the external air temperature and the temperature of the cartridge 2000 are maintained at room temperature, and the surface temperature of the target region before refrigerant spraying is about 36.5° C.). The initial time Ti may be determined, for example, within 1 second, and preferably within 0.5 seconds, but is not limited thereto.

Referring to (b) of FIG. 6, it can be seen that in terms of a surface temperature change amount during the initial time Ti, the first control mode may exhibit a smaller surface temperature change amount than the second control mode or the third control mode. This is because the magnitude of voltage, current, or power applied to the temperature controller 1300 in the initial heating period P1 in the first control mode is larger than that in the second control mode or the third control mode.

The second control mode may have a different feedback control start condition than the third control mode. For example, when the feedback control start condition for the second control mode is that a first time elapses from the time point at which refrigerant spraying is started, and for the third control mode is that a second time elapses from the time point at which refrigerant spraying is started, the first time may shorter than the second time. For another example, when the feedback control start condition for the second control mode is that a target temperature of the target region reaches a first temperature, and for the third control mode is that a target temperature of the target region reaches a second temperature, the first temperature may be higher than the second temperature.

Therefore, when performing the cooling procedure on the substantially same target in the substantially same environment, a feedback control start time point after the time point at which refrigerant spraying is started in the second control mode may be earlier than that in the third control mode.

In the initial heating period P1, the magnitude of voltage, current, or power applied to the temperature controller 1300 according to the second control mode may be substantially the same as that of voltage, current, or power applied to the temperature controller 1300 according to the third control mode. Alternatively, the amount of heat energy applied to the refrigerant per unit time in the initial heating period P1 may be substantially the same in both the second control mode and the third control mode.

Meanwhile, when the refrigerant spraying device 1000 is operated according to the second control mode, a time point at which voltage, current, or power is applied to the temperature controller 1300 may be faster compared to when the refrigerant spraying device 1000 is operated according to the third control mode. For example, a first standby time from the time point at which refrigerant spraying is started by the flow regulator 1400 to the time point at which voltage, current, or power is applied to the temperature controller 1300 when the refrigerant spraying device 1000 is operated according to the second control mode may be shorter than a second standby time from the time point at which refrigerant spraying is started by the flow regulator 1400 to the time point at which voltage, current, or power is applied to the temperature controller 1300 when the refrigerant spraying device 1000 is operated according to the third control mode.

Referring to (a) of FIG. 6 and (b) of FIG. 6, in the second control mode and the third control mode, the initial cooling rate may be substantially the same, but the time taken for the surface temperature of the target region to reach the target cooling temperature may be different.

When comparing the cooling rate, the cooling rate may be faster in the order of the first control mode, the second control mode, and the third control mode. In other words, the time taken for the surface temperature of the target region to reach the target cooling temperature may decrease in the order of the first control mode, the second control mode, and the third control mode. Referring to (a) of FIG. 6, when the cooling procedure is performed on the substantially same target in the substantially same environment, the time taken for the surface temperature of the target region to reach the target cooling temperature is a first arrival time a for the first control mode, a second arrival time b for the second control mode, and a third arrival time c for the third control mode, the first arrival time a may be shorter than the second arrival time b and the second arrival time b may be shorter than the third arrival time c.

Meanwhile, when considering procedure safety, it may be understood that the safety is high in an order of the third control mode, the second control mode, and the first control mode. This is because, since the initial cooling rate in the first control mode is slower than that in the second or third control mode, so that a possibility of the surface temperature of the target region reaching a temperature that causes irreversible damage is low. This is also because, since feedback control in the second control mode starts earlier than in the third control mode, so that a possibility of the surface temperature of the target region reaching a temperature that causes irreversible damage is low.

In the above, the first to third control modes have been described, but the technical idea of the present disclosure is not limited thereto, and various types of control modes may exist depending on the amount of heat energy or the amount of heat energy per unit time transferred to the refrigerant from the temperature controller 1300 in the initial heating period P1 and the feedback control initiation condition. These control modes may be distinguished from each other based on the initial cooling rate and/or the time taken for the target region to reach the target cooling temperature. For example, a plurality of control modes may be graded according to the initial cooling rate, which may mean that an individual control mode is designed for each step.

### Use of Step-by-Step Control Method

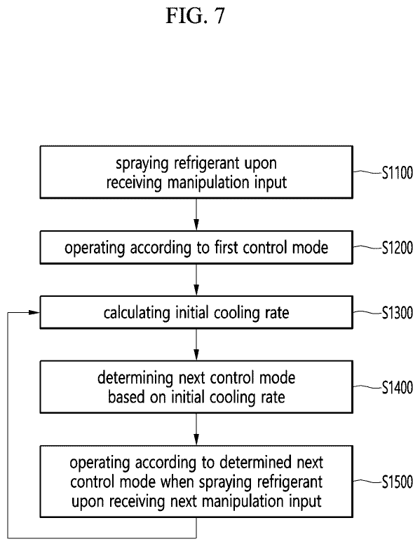

Hereinafter, a method of controlling a cooling rate by controlling the refrigerant spraying device 1000 using the step-by-step control method will be described with reference to FIGS. 7 to 9.

Hereinafter, for convenience of explanation, a case of designing a method of controlling a cooling rate using the first to third control modes described with reference to FIG. 6 will be described. However, the technical idea of the present disclosure is not limited thereto, at least one other control mode may be further used in addition to the first to third control modes.

The basic principle of the method of controlling the cooling rate is to determine a control mode of the temperature controller 1300 in a next shot using information in a previous shot. For example, a control mode in a current shot may be determined by comparing the initial cooling rate and a threshold range in the previous shot, and a control mode in the next shot may be determined by comparing the initial cooling rate and the threshold range in the current shot.

Here, the shot may refer to an operation from the start to stop of refrigerant spraying. Specifically, one shot may be defined as an operation in which the flow regulator 1400 is controlled to start refrigerant spraying to an operation in which the flow regulator 1400 is controlled to stop refrigerant spraying. However, the technical idea of the present disclosure is not limited thereto, and shots may be divided depending on power on/off of the refrigerant spraying device 1000, a time point at which the setting of the target cooling temperature is changed, and/or a time point at which the target cooling time is changed.

In addition, different shots may be performed on different target regions. For example, a first shot may be performed on a first target region, and a second shot may be performed on a second target region that is different from the first target region. However, different shots may also be performed on a same or substantially same target region.

In addition, different shots may be performed in different time periods. For example, the first shot may be performed as a first manipulation input is received in a first time period, the second shot may be performed as a second manipulation input is received in a second time period, and the second time period may begin after the end of the first time period.

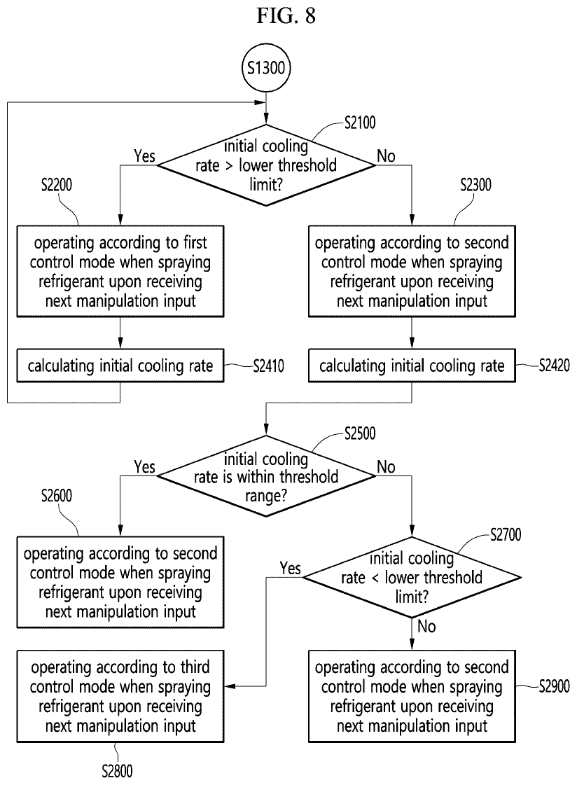

FIGS. 7 and 8 are flowcharts illustrating a method of controlling a cooling rate according to an embodiment.

Referring to FIG. 7, the method of controlling the cooling rate may include spraying the refrigerant upon receiving a manipulation input (S1100), operating by the first control mode (S1200), calculating the initial cooling rate (S1300), determining a next control mode based on the initial cooling rate (S1400), and operating by the determined next control mode when spraying the refrigerant upon receiving a next manipulation input (S1500).

Meanwhile, prior to the method of controlling the cooling rate to be described with reference to FIG. 7, turning on power of the refrigerant spraying device 1000 and setting the target cooling temperature and/or target cooling time may be performed. In other words, the method of controlling the cooling rate may be understood as being performed while the refrigerant spraying device 1000 prepares to start refrigerant spraying.

Hereinafter, each step of the method of controlling the cooling rate will be described in detail.

First, the refrigerant spraying device 1000 may spray the refrigerant upon receiving the manipulation input (S1100). For example, the controller 1900 may receive the first manipulation input from the user and control the flow regulator 1400 to spray the refrigerant. Here, the first manipulation input may be an operation of temporarily pressing a push button switch included in the input unit 1700.

The temperature controller 1300 may be operated according to the first control mode (S1200). Specifically, the controller 1900 may load the first control mode among the plurality of control modes stored in the memory of the refrigerant spraying device 1000 and apply voltage, current, or power to the temperature controller 1300 according to the first control mode.

Here, the first control mode may be understood as the safest control mode among the plurality of control modes. For example, according to the first control mode, the magnitude of voltage, current, or power applied to the temperature controller 1300 in the initial heating period P1 may be largest compared to other control modes.

When performing the cooling procedure on the substantially same target in the substantially same environment, the first control mode may have the following characteristics when compared to other control modes.

According to the first control mode, the amount of heat energy per unit time transferred to the refrigerant until the surface temperature of the target region reaches the target cooling temperature may be the largest compared to other control modes. Alternatively, according to the first control mode, the total amount of heat energy transferred to the refrigerant until the surface temperature of the target region reaches the target cooling temperature may be the largest compared to other control modes.

According to the first control mode, the amount of heat energy transferred to the refrigerant per unit time before feedback control is started may be the largest compared to other control modes. Alternatively, according to the first control mode, the total amount of heat energy transferred to the refrigerant before feedback control is started may be the largest compared to other control modes.

Alternatively, according to the first control mode, the amount of heat energy transferred to the refrigerant per unit time during the initial time Ti after the start of refrigerant spraying may be the largest compared to other control modes. Alternatively, according to the first control mode, the total amount of heat energy transferred to the refrigerant during the initial time Ti after the start of refrigerant spraying may be the largest compared to other control modes.

Meanwhile, the amount of heat energy per unit time described above may correspond to the amount of power applied to the temperature controller 1300, and the total amount of heat energy may correspond to the amount of power applied to the temperature controller 1300.

As described above, the first control mode is the safest control mode among the plurality of control modes, and may be understood as the basic control mode for the refrigerant spraying device 1000. The basic control mode may be understood as a control mode used when operating the refrigerant spraying device 1000 for the first time, or when changing or resetting control variable settings of the refrigerant spraying device 1000. As such, by using the safest first control mode as the basic control mode, safety for patients may be secured regardless of environmental conditions in which the cooling procedure is performed.

Meanwhile, a time point at which the temperature controller 1300 is started to be controlled by the first control mode may be correspond to the time point at which refrigerant spraying is started. However, the technical idea of the present disclosure is not limited thereto, and the controller 1900 may control the temperature controller 1300 by loading the first control mode before the time point at which refrigerant spraying is started. Specifically, when the first shot is performed within the first time period, the temperature controller 1300 may be controlled according to the first control mode in the first time period. In other words, step S1200 may be performed not only while step S1100 is performed or after step S1100 is performed, but also before step S1100 is performed.

In the process of operating by the first control mode, the initial cooling rate may be calculated (S1300). For example, in the first shot performed upon receiving the first manipulation input, a first initial cooling rate during the initial time Ti after the time point at which refrigerant spraying is started may be calculated. More specifically, the controller 1900 may acquire the first surface temperature of the first target region corresponding to the first time point at which refrigerant spraying is started by using the sensor unit 1600, acquire the second surface temperature of the first target region corresponding to the second time point after the initial time Ti elapses from the first time point, and calculate the first initial cooling rate by dividing the difference between the first surface temperature and the second surface temperature by the initial time Ti.

Meanwhile, the method for calculating the initial cooling rate is not limited to the above-described method. For example, the initial cooling rate may be calculated based on the second time point after the initial time Ti elapses from the first time point after a predetermined of time from the time point at which refrigerant spraying is started.

The next control mode may be determined based on the initial cooling rate (S1400).

As an example, the controller 1900 may maintain or change the control mode by considering the initial cooling rate and the threshold range. Specifically, the controller 1900 may maintain the control mode when the initial cooling rate is within the threshold range. Alternatively, the controller 1900 may change the control mode when the initial cooling rate is out of the threshold range.

Here, the threshold range is a range in which irreversible damage to the target region is prevented and the cooling rate is equal or higher than a predetermined level, and may be determined through experiment. The threshold range may be defined as a lower threshold limit and an upper threshold limit. In addition, the threshold range may be set differently for each shot. Alternatively, the threshold range may be set differently depending on the current control mode.

As another example, the controller 1900 may maintain or change the control mode by considering the initial cooling rate and a plurality of threshold ranges. Specifically, a threshold range for each control mode may be predetermined and stored in the memory, and the controller 1900 may determine a control mode corresponding to the threshold range to which the initial cooling rate belongs as the next control mode.

Meanwhile, in a case where the control mode is changed, when the current control mode is the first control mode, the changed control mode may be the second control mode. As described above, the second control mode may exhibit a faster cooling rate than the first control mode and a slower cooling rate than the third control mode. The change from the first control mode to the second control mode may be understood as an attempt to increase the cooling rate step by step, thereby preventing damage to the target region and enabling a safer procedure. Meanwhile, when the current control mode is the second control mode, the control mode may be changed to the first control mode or the second control mode, and when the current control mode is the third control mode, the control mode may be changed to the second control mode.

In the above, it has been described that the control mode is determined using the initial cooling rate in step S1400, but the technical idea of the present disclosure is not limited thereto. For example, the control mode may be determined using an initial temperature difference. Here, the initial temperature difference may refer to a difference between the first surface temperature of the target region corresponding to the first time point at which refrigerant spraying is started and the second surface temperature of the target region corresponding to the second time point after the initial time Ti elapses from the first time point.

The refrigerant spraying device 1000 may be operated according to the next control mode determined in step S1400 when spraying the refrigerant upon receiving the next manipulation input (S1500). For example, the controller 1900 may maintain the control mode as the first control mode when the first initial cooling rate calculated in the first shot is within the threshold range, and apply voltage, current, or power applied to the temperature controller 1300 according to the first control mode in the second shot after the first shot. Alternatively, the controller 1900 may change the control mode to the second control mode when the first initial cooling rate calculated in the first shot is out of the threshold range, and apply voltage, current, or power applied to the temperature controller 1300 according to the second control mode in the second shot after the first shot.

A control mode corresponding to a next shot may be determined based on the initial cooling rate calculated in a shot performed in step S1500. For example, the control mode in the second shot after the first shot may be determined based on the first initial cooling rate calculated in the first shot, and a control mode in a third shot after the second shot may be determined based on a second initial cooling rate calculated in the second shot. In other words, as illustrated in FIG. 7, step S1300 may be performed after step S1500, and steps S1300, S1400, and S1500 may be repeated for each shot. At this time, the first shot may be performed on the first target region, the second shot may be performed on the second target region, the third shot may be performed on a third target region, and the first to third target regions may be different target regions. However, the first target region and the second target region may be a substantially same region, the second target region and the third target region may be a substantially same region, the third target region and the first target region may be a substantially same region, or the first to third target regions may be a substantially same region.

Hereinafter, the method of controlling the cooling rate will be described in more detail with reference to FIG. 8. In particular, an example comparing the initial cooling rate and the threshold range described with reference to FIG. 7 and a case where the first control mode, the second control mode, and the third control mode are individually performed will be described. For convenience of explanation, it is assumed that steps S1100, S1200, and S1300 described with reference to FIG. 7 have been performed, and steps performed after step S1300 will be described.

Referring to FIG. 8, first, it may be determined whether the initial cooling rate is larger than the lower threshold limit (S2100). For example, the controller 1900 may determine whether the first initial cooling rate in the first shot is larger than the lower threshold limit. Here, the lower threshold limit may refer to a lower limit of the above-described threshold range.

When the initial cooling rate is larger than the lower threshold limit, it may mean that the initial cooling rate is within the threshold range or larger than the upper threshold limit of the threshold range. When the initial cooling rate is within the threshold range, it needs to be maintained. This is because the first control mode may be understood as an appropriate control mode. Meanwhile, when the initial cooling rate is equal to or larger than the upper threshold limit, a change needs to be made to a control mode with a slower cooling rate. This is because this means that the cooling rate is relatively fast. However, since the first control mode corresponds to the control mode with the slowest cooling rate, the control mode does not need to be changed from the first control mode even when the initial cooling rate is equal to or larger than the upper threshold limit.

Therefore, in a case where the current control mode is the safest first control mode, when the initial cooling rate is larger than the lower threshold limit, the refrigerant spraying device 1000 may be operated by the first control mode when spraying the refrigerant upon receiving the next manipulation input (S2200).

Meanwhile, when the initial cooling rate is less than the lower threshold limit, it means that the cooling rate is relatively slow, so a change needs to be made to a control mode with a faster cooling rate.

Therefore, in a case where the current control mode is the first control mode, when the initial cooling rate is less than the lower threshold limit, the refrigerant spraying device 1000 may be operated by the second control mode when spraying the refrigerant upon receiving the next manipulation input (S2300).

After step S2200, the initial cooling rate may be calculated (S2410). Step S2200 may refer to a process in which a new second shot is performed, and step S2410 may be understood as a step of calculating the second initial cooling rate in the second shot.

Likewise, after step S2300, the initial cooling rate may be calculated (S2420). Step S2300 may also mean a process in which a new second shot is performed, and step S2420 may be understood as a step of calculating the second initial cooling rate in the second shot.

Meanwhile, as illustrated in FIG. 8, step S2100 may be performed again after step S2410, while step S2500 may be performed after step S2420. This is because, in step S2410, the refrigerant spraying device 1000 is controlled according to the first control mode, while in step S2420, the refrigerant spraying device 1000 is controlled according to the second control mode. In other words, the algorithm for determining the control mode in the next shot may vary depending on the control mode by which the refrigerant spraying device 1000 is currently controlled. When steps S2100 to S2300 are referred to as a first algorithm for determining the control mode in the next shot, steps S2500 to S2900, which will be described later, may be understood as a second algorithm.

The second algorithm is structured as follows.

First, it may be determined whether the initial cooling rate is within the threshold range (S2500). For example, the controller 1900 may determine whether the second initial cooling rate in the second shot is within threshold range.

When the initial cooling rate is within the threshold range, the refrigerant spraying device 1000 may be operated by the second control mode when spraying the refrigerant upon receiving the next manipulation input (S2600). For example, when the second initial cooling rate in the second shot in which the temperature controller 1300 is operated according to the second control mode is within the threshold range, the temperature controller 1300 may be operated according to the second control mode in the third shot after the second shot. This may be understood that the control mode is maintained because the initial cooling rate is within an appropriate range.

Meanwhile, when the initial cooling rate is out of the threshold range, it may be determined whether the initial cooling rate is less than the lower threshold limit (S2700). For example, the controller 1900 may determine whether the second initial cooling rate is less than the lower threshold limit.

When the initial cooling rate is less than the lower threshold limit, the refrigerant spraying device 1000 may be operated by the third control mode when spraying the refrigerant upon receiving the next manipulation input (S2800). For example, when the second initial cooling rate in the second shot in which the temperature controller 1300 is operated by the second control mode is less than the lower threshold limit, the temperature controller 1300 may be operated by the third control mode in the third shot after the second shot. This may be understood that the temperature controller 1300 needs to be controlled by the third control mode having a faster cooling rate than the second control mode because the initial cooling rate is slower than an appropriate rate.

Meanwhile, the initial cooling rate is not less than the lower threshold limit, the refrigerant spraying device 1000 may be operated by the first control mode when spraying the refrigerant upon receiving the next manipulation input (S2900). For example, when the second initial cooling rate in the second shot in which the temperature controller 1300 is operated by the second control mode is not within the threshold range and is not less than the lower threshold limit (i.e., the second initial cooling rate is larger than the upper threshold limit), the temperature controller 1300 may be operated according to the first control mode in the third shot after the second shot. This may be understood that the temperature controller 1300 needs to be controlled according to the first control mode having a slower cooling rate than the second control mode because the initial cooling rate is faster than an appropriate rate.

Meanwhile, a time point to determine whether to change or maintain the control mode may be between a time point at which the initial cooling rate is calculated in the previous shot and a time point at which the current shot is started. For example, the control mode of the temperature controller 1300 in the second shot after the first shot may be determined between a time point at which the first initial cooling rate is calculated in the first shot and a time point at which refrigerant spraying is started in the second shot. For another example, the control mode of the temperature controller 1300 in the second shot after the first shot may be determined between the time point at which the first initial cooling rate is calculated in the first shot and a time point at which the first shot is ended.

In the above, it has been mainly described that the control mode is changed based on the previous shot, but the technical idea of the present disclosure is not limited thereto.

As an example, the control mode of the temperature controller 1300 may be determined based on the external temperature of the refrigerant spraying device 1000 and/or the temperature of the cartridge 2000 for each shot. Specifically, the control mode of the temperature controller 1300 may be determined based on various measurement variables, such as the external air temperature immediately before performing each shot, the refrigerant temperature outside or inside the cartridge 2000, the internal pressure of the cartridge 2000, and the surface temperature of the target region. At this time, the control mode of the temperature controller 1300 does not need to start from the first control mode and change to the second control mode and the third control mode sequentially, and may start from the second control mode or the third control mode and change to the first control mode in subsequent shots. Meanwhile, when determining the control mode based on the various measurement variables described above, it goes without saying that the initial cooling rate in the previous shot described above may be further taken into account.

Control Using Basic Control Mode when Reset Conditions are Satisfied

Meanwhile, the refrigerant spraying device 1000 may need to be controlled using the safest first control mode depending on cases. Specifically, when a predetermined time elapses after the first shot is performed using the refrigerant spraying device 1000, the cartridge 2000 is replaced, or a target cooling temperature or target cooling time is newly set, the procedure environment may change. Therefore, the refrigerant spraying device 1000 needs to be operated according to the safest control mode.

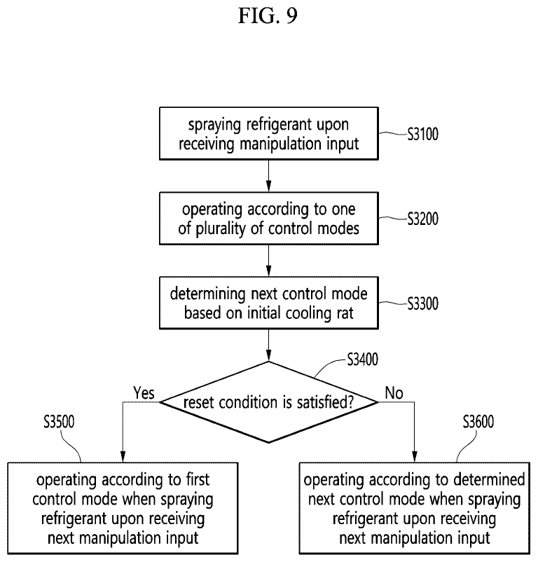

Hereinafter, a reset process of resetting the control mode of the refrigerant spraying device 1000 will be described with reference to FIG. 9.

Referring to FIG. 9, the reset process may include spraying the refrigerant upon receiving a manipulation input (S3100), operating according to any one of a plurality of control modes (S3200), determining a next control mode based on the initial cooling rate (S3300), determining whether a reset condition is satisfied (S3400), operating according to the first control mode when spraying the refrigerant upon receiving a next manipulation input (S3500), and operating according to the determined next control mode when spraying the refrigerant upon receiving the next manipulation input (S3600).

Hereinafter, each step will be described in detail.

First, the refrigerant spraying device 1000 may spray the refrigerant upon receiving the manipulation input (3100). The description of step S1100 made above may be applicable to step S3100, and thus the above description will not be repeated.

The refrigerant spraying device 1000 may be operated according to any one of the plurality of control modes. For example, the refrigerant spraying device 1000 may be operated according to any one of the first to third control modes described above.

The next control mode may be determined based on the initial cooling rate (S3300). For example, the controller 1900 may calculate the initial cooling rate in a shot performed in steps S3100 and S3200, and determine a control mode corresponding to a next shot based on the calculated initial cooling rate. The description of step S1400 made above with reference to FIG. 7 may be applicable to the step of determining the next control mode. Alternatively, the description of the first algorithm or the second algorithm made above with reference to FIG. 8 may be applicable to the step of the determining the next control mode.

It may be determined whether the reset condition is satisfied (S3400). For example, the controller 1900 may determine whether the reset condition is satisfied, and perform step S3500 or step S3600, which will be described later, depending on whether the reset condition is satisfied.

The reset condition may include various conditions as follows.

As an example, the reset condition may include a condition in which a reset time elapses while the refrigerant is not sprayed. Specifically, the reset condition may include a condition in which the reset time elapses from a time point at which the flow regulator 1400 blocks movement of the refrigerant.

Here, the reset time may be determined within 60 seconds. Preferably, the reset time may be 30 seconds. The reset time may be determined based on an average time taken for the user to replace the cartridge 2000. A replacement time for replacing the cartridge 2000 may be derived experimentally. When replacing the cartridge 2000, a change in temperature of the refrigerant may increase depending on the temperature or internal pressure of a new cartridge 2000. Therefore, the control mode of the refrigerant spraying device 1000 needs to be reset and set to the first control mode, which is the safest control mode.

As another example, the reset condition may include a condition in which power of the refrigerant spraying device 1000 is turned off. For example, when the refrigerant spraying device 1000 is turned off and then turned on again, it may be determined that the reset condition is satisfied.

As another example, the reset condition may include a condition in which a state of the refrigerant spraying device 1000 is changed from a preparation state. For example, in a state where the refrigerant spraying device 1000 enters a spraying preparation state to prepare to start refrigerant spraying after the target cooling temperature and the target cooling time are set, when a variable setting state for setting the target cooling temperature and the target cooling time is entered again, it may be determined that the reset condition is satisfied. At this time, the refrigerant spraying device 1000 may enter the variable setting state when a manipulation signal is received by the input unit 1700 in the spraying preparation state, and when the manipulation signal is received, it may be determined that the reset condition is satisfied.

When any one of the above-described conditions is satisfied, the reset condition may be determined to be satisfied. However, the technical idea of the present disclosure is not limited thereto, and the reset condition may include only one of the above-described conditions.

When the above-described reset condition is satisfied, the refrigerant spraying device 1000 may be operated by the first control mode when spraying the refrigerant in response to the next operation input (S3500). For example, in a case where the control mode corresponding to the second shot is determined to be the first control mode based on the first initial cooling rate in the first shot, when the reset condition is satisfied, the control mode of the refrigerant spraying device 1000 may be maintained as the first control mode in the second shot. For another example, in a case where the control mode corresponding to the second shot is determined to be the second control mode or the third control mode based on the first initial cooling rate in the first shot, when the reset condition is satisfied, the refrigerant spraying device 1000 may be operated by the first control mode in the second shot.

When the reset condition is not satisfied, the refrigerant spraying device 1000 may be operated by the determined next control mode when spraying the refrigerant upon receiving the next operation input (S3600). Here, the determined next control mode may be understood as the next control mode determined in step S3300.

Meanwhile, step S3400 may be performed before step S3300. At this time, when the reset condition is satisfied in step S3400, step S3300 may be omitted, and when the reset condition is not satisfied in step S3400, step S3300 may be performed and then step S3600 may be performed.

The features, structures, effects, and the like described in the embodiments are included in at least one embodiment of the present disclosure and are not necessarily limited to one embodiment. Furthermore, the features, structures, effects, and the like provided in each embodiment can be combined or modified in other embodiments by those skilled in the art to which the embodiments belong. Therefore, the contents related to the combination and modification should be construed to be included in the scope of the present disclosure.

The embodiments described herein have been provided merely for illustrative purposes, those skilled in the art will appreciate that various modifications, additions, and substitutions are possible, without departing from the scope and spirit of the present disclosure. In other words, each element specifically shown in the embodiments can be implemented in modified forms. Therefore, the contents related to the combination and modification should be construed to be included in the scope of the present disclosure as disclosed in the accompanying claims.

What is claimed is:

1. A coolant spraying device for spraying a coolant such that a temperature of a target region is maintained at a target temperature, the coolant spraying device comprising:
a flow regulator configured to allow or block spraying of the coolant;
a nozzle configured to spray the coolant that has passed through the flow regulator;
a tube configured to provide a flow path through which the coolant flows;
a heater thermally coupled to the tube and configured to heat the tube such that heat generated by the heater is conducted to the coolant passing through the tube;
a temperature sensor configured to measure at least part of the target region; and
a controller configured to control the flow regulator and the heater,
wherein, in response to the controller determining to start a new cooling treatment procedure, the controller is configured to:
control the flow regulator to spray the coolant,
control an electrical power supplied to the heater according to an initial control mode selected among a plurality of pre-stored initial control modes,
wherein the plurality of pre-stored initial control modes comprises a first initial control mode and a second initial control mode, and
wherein a first electrical power supplied according to the first initial control mode is greater than a second electrical power supplied according to the second initial control mode,
in response to a measured temperature by the temperature sensor reaching a predetermined temperature which is higher than the target temperature, control an electrical power supplied to the heater according to a feedback control method, based on the measured temperature and the target temperature, to maintain the measured temperature at the target temperature,
wherein a current initial control mode corresponding to a current cooling treatment procedure is configured to be selected based on a past initial cooling rate calculated during at least part of a time period during which the controller controls an electrical power supplied to the heater according to a past initial control mode corresponding to a past cooling treatment procedure, and
wherein the past cooling treatment procedure is a previous treatment procedure of the current cooling treatment procedure.

2. The coolant spraying device of claim 1, wherein the current initial control mode is configured to be applied when the current cooling treatment procedure is started.

3. The coolant spraying device of claim 2, wherein the applying of current initial control mode is configured to be terminated when performing of the feedback control method is started in response to the measured temperature by the temperature sensor reaching the predetermined temperature.

4. The coolant spraying device of claim 1, wherein the past initial control mode is configured to be applied when the past cooling treatment procedure is started.

5. The coolant spraying device of claim 4, wherein the applying of past initial control mode is configured to be terminated when performing of the feedback control method is started in response to the measured temperature by the temperature sensor reaching the predetermined temperature.

6. The coolant spraying device of claim 1, wherein the first initial control mode is configured to be selected as the current initial control mode in response to the past initial cooling rate being lower than a lower threshold and the past initial cooling rate being the second initial control mode, or
the second initial control mode is configured to be selected as the current initial control mode in response to the past initial cooling rate being higher than an upper threshold and the past initial cooling rate being the first initial control mode.

7. The coolant spraying device of claim 1, wherein the past initial control mode is configured to be selected as the current initial control mode in response to the past initial cooling rate being between a lower threshold and an upper threshold.

8. The coolant spraying device of claim 1, wherein the first initial control mode is configured to be selected as an initial control mode in response to reset condition being satisfied.

9. The coolant spraying device of claim 8, wherein the reset condition includes at least one of (i) expiration of a reset timer after the flow regulator has been closed, (ii) power-on of the coolant spraying device after power-off of the coolant spraying device, (iii) reconfiguration of the target temperature and/or a target cooling time or (iv) replacement of a cartridge storing the coolant.

10. The coolant spraying device of claim 1, wherein the controller is configured to determine to start the new cooling treatment procedure in response to a trigger input being received.

11. A coolant spraying device for spraying a coolant such that a temperature of a target region is maintained at a target temperature, the coolant spraying device comprising:
a flow regulator configured to allow or block spraying of the coolant;
a nozzle configured to spray the coolant that has passed through the flow regulator;
a tube configured to provide a flow path through which the coolant flows;
a heater thermally coupled to the tube and configured to heat the tube such that heat generated by the heater is conducted to the coolant passing through the tube;
a temperature sensor configured to measure at least part of the target region; and
a controller configured to control the flow regulator and the heater,
wherein, in response to the controller determining to start a new cooling treatment procedure, the controller is configured to:
control the flow regulator to spray the coolant, and
in response to a measured temperature by the temperature sensor reaching a predetermined temperature corresponding to an initial control mode selected among a plurality of pre-stored initial control modes, control an electrical power supplied to the heater according to a feedback control method, based on the measured temperature and the target temperature, to maintain the measured temperature at the target temperature, the predetermined temperature being higher than the target temperature,
wherein each pre-stored initial control mode includes a temperature difference between a target temperature and a predetermined temperature,
wherein the plurality of pre-stored initial control modes comprise a first initial control mode and a second initial control mode, and
wherein a first temperature difference included in the first initial control mode is higher than a second temperature difference included in the second initial control mode,
wherein a current initial control mode corresponding to a current cooling treatment procedure is configured to be selected based on a past initial cooling rate calculated during at least part of a time period between a start time of spraying the coolant and a time of the measured temperature reaching the predetermined temperature, and
wherein the past cooling treatment procedure is a previous treatment procedure of the current cooling treatment procedure.

12. The coolant spraying device of claim 11, wherein the current initial control mode is configured to be applied when the current cooling treatment procedure is started.

13. The coolant spraying device of claim 12, wherein the applying of current initial control mode is configured to be terminated when performing of the feedback control method is started in response to the measured temperature by the temperature sensor reaching the predetermined temperature.

14. The coolant spraying device of claim 11, wherein the past initial control mode is configured to be applied when the past cooling treatment procedure is started.

15. The coolant spraying device of claim 14, wherein the applying of past initial control mode is configured to be terminated when performing of the feedback control method is started in response to the measured temperature by the temperature sensor reaching the predetermined temperature.

16. The coolant spraying device of claim 11, wherein the first initial control mode is configured to be selected as the current initial control mode in response to the past initial cooling rate being lower than a lower threshold and the past initial cooling rate being the second initial control mode, or
the second initial control mode is configured to be selected as the current initial control mode in response to the past initial cooling rate being higher than an upper threshold and the past initial cooling rate being the first initial control mode.

17. The coolant spraying device of claim 11, wherein the past initial control mode is configured to be selected as the current initial control mode in response to the past initial cooling rate being between a lower threshold and an upper threshold.

18. The coolant spraying device of claim 11, wherein the first initial control mode is configured to be selected as an initial control mode in response to reset condition being satisfied.

19. The coolant spraying device of claim 18, wherein the reset condition includes at least one of (i) expiration of a reset timer after the flow regulator has been closed, (ii) power-on of the coolant spraying device after power-off of the coolant spraying device, (iii) reconfiguration of the target temperature and/or a target cooling time or (iv) replacement of a cartridge storing the coolant.

20. The coolant spraying device of claim 11, wherein the controller is configured to determine to start the new cooling treatment procedure in response to a trigger input being received.
